(19) United States
(12) Patent Application Publication
(10) Pub. No.: US 2026/0207627 A1
(43) Pub. Date: Jul. 23, 2026

(54) PHYTOECDYSONES FOR USE IN THE TREATMENT OF INFLAMMATORY RESPIRATORY DISEASES

(71) Applicant: BIOPHYTIS, PARIS (FR)

(72) Inventors: Mathilde LATIL, PARIS (FR); Pierre DILDA, PARIS (FR); Stanislas VEILLET, SAVIGNY SUR ORGE (FR)

(21) Appl. No.: 19/141,106

(22) PCT Filed: Dec. 21, 2023
(86) PCT No.: PCT/FR2023/052080

§ 371 Date: Jun. 19, 2025

(30) Foreign Application Priority Data
Dec. 23, 2022 (FR) FR2214426

(51) Int. Cl. A61K 31/575; A61K 36/21; A61K 36/28; A61K 36/88; A61P 11/06

(52) U.S. Cl. CPC A61K 31/575; A61K 36/21; A61K 36/28; A61K 36/88; A61P 11/06

(57) ABSTRACT

The invention relates to a composition including at least one phytoecdysone and/or at least one semi-synthetic derivative of phytoecdysone, for use in the treatment of an inflammatory respiratory disease in mammals.

### CROSS REFERENCE TO RELATED APPLICATIONS

This application is a National Stage of International Application No. PCT/FR2023/052080, having an International Filing Date of 21 Dec. 2023, which designated the United States of America, and which International Application was published under PCT Article 21(2) as WO Publication No. 2024/134105, which claims priority from and the benefit of French Patent Application No. 2214426 filed on 23 Dec. 2022, the disclosures of which are incorporated herein by reference in their entireties.

### BACKGROUND

### Field

The disclosure relates to treatments of inflammatory respiratory diseases. It falls within the field of medicinal products/dietary supplements for such treatments and relates more particularly to phytoecdysones intended for the prevention and/or treatment of inflammatory respiratory diseases.

### BRIEF DESCRIPTION OF RELATED DEVELOPMENTS

Phytoecdysones are an important family of polyhydroxylated plant sterols structurally similar to insect molting hormones. These molecules are produced by many plant species and help defend them against insect pests. The most common phytoecdysone is 20-hydroxyecdysone (20E). 20E is pharmacologically active in mammals. It activates the Mas receptor, a receptor of the protective arm of the Renin-Angiotensin system (Lafont et al., 2021) which induces a number of beneficial effects which have been described in preclinical studies in normal and pathological contexts.

BI0101 is an oral preparation of 20-hydroxyecdysone with a purity greater than or equal to 97%. Its preparation method is disclosed in international patent application WO2018197731 (Lafont et al. 2018). BI0101 is a new drug candidate clinically developed in the treatment of sarcopenia, Duchenne muscular dystrophy and COVID-19. These last two therapeutic applications are the subject of international patent applications WO2018197708 (Dilda et al. 2018) and WO2021198588 (Dilda et al. 2021). Semi-synthetic derivatives of 20-hydroxyecdysone have also been developed, as disclosed in international patent application WO2015177469 (Lafont et al. 2015), and are used for such therapeutic applications.

Asthma is a chronic inflammatory disease of the respiratory tract characterized by chronic deregulated inflammation, and exacerbated bronchial reactivity. It is the most common respiratory disease worldwide, and its prevalence has increased significantly in recent decades, reaching 5 to 10%, and affecting 339 million people worldwide (Bloom et al., 2019; Global burden of 369 diseases and injuries in 204 countries and territories, 1990-2019: a systematic analysis for the Global Burden of Disease Study 2019). Asthma is defined by variable respiratory symptoms such as shortness of breath, difficulty breathing (dyspnea), wheeze, tightness of chest and cough, associated with chronic bronchial inflammation (Redell et al., 2015). Airflow restriction and the presence of mucus cause real breathing difficulties. The frequency of attacks (hours or days apart, or even months apart) and their severity vary from patient to patient. Symptoms may worsen with physical exertion or at night.

Certain risk factors are involved in the onset of an asthma which is the combination of a genetic predisposition and environmental factors such as allergens present inside homes (house dust mites, mold), outdoor allergens (pollen), cigarette smoke, or air pollution (fine particles).

The physiopathological characteristics of asthma can be divided into three interdependent components: exacerbated bronchial airway reactivity, airway remodeling, and airway inflammation.

Airway smooth muscle cells contribute to the pathogenesis of asthma, mainly through their contractile properties (Mims, 2015). Indeed, exacerbated bronchial airway reactivity is defined by an excessive response to harmless or harmful stimuli. This impaired bronchial response to the environment is dependent on the activity of the bronchial smooth muscle, the main participant in bronchoconstriction. The degree of increased airway reactivity is correlated with asthma severity and the need for treatment.

Beyond contractile considerations, active involvement of the airway smooth muscle in bronchial remodeling mechanisms is now established (Boulet, 2018). Airway remodeling includes alterations of the epithelium with mucus cell hyperplasia and epithelial cell fragility, subepithelial fibrosis with extracellular matrix modifications, bronchial smooth muscle cell hypertrophy, and bronchial submucosal blood vessel, nerve and gland hyperplasia. Cytokines and growth factors, such as FGF or TGFβ, but also extracellular matrix composition have been described as powerful airway smooth muscle cell phenotype modifiers. These abnormalities are associated with asthma chronicity and severity.

Regarding airway inflammation, two main physiopathological pathways are commonly accepted: eosinophilic asthma (or type 2 asthma) and non-eosinophilic asthma. In type 2 asthma, the main cytokines involved are interleukin 4, involved in T helper 2 cell polarization and switching to immunoglobins E (IgE), interleukin 5 associated with eosinophil production and trafficking, and finally, interleukin 13, which plays a central role in airway remodeling. Conversely, non-eosinophilic asthma remains poorly understood (Habib et al., 2022; Carr et al., 2018).

The majority of asthma patients are well-controlled with short-acting or long-acting beta-agonist treatments and also with inhaled corticosteroids, antileukotrienes, anticholinergics, theophylline or biotherapies such as anti-IgE (omalizumab), anti-IL-5 (mepolizumab, reslizumab, and benralizumab) or anti-IL-4 receptor (dupilumab).

Conventional asthma treatments mainly target two physiopathological mechanisms: inflammation and bronchoconstriction. For example, inhaled bronchodilators (beta-2 agonists and anticholinergics) target airway smooth muscle cells directly by reducing their contractility to improve airflow and reduce chronic and acute symptoms. Inhaled corticosteroids, on the other hand, mainly target inflammation but also affect contractility and also the proliferation of airway smooth muscle cells (Goldsmith et al., 2007; Goto et al., 2008).

However, severe asthma, the most serious level of the disease, is uncontrolled in terms of chronic symptoms, exacerbations, permanent bronchial obstruction, which persist in spite of maximum management, which requires continuous use of short-acting bronchodilators in spite of maximum inhaled corticosteroid doses. This results in patients having a poor quality of life and also substantial direct care costs (care visits and treatment), and also indirect costs (working day).

Although recent biotherapies have significantly improved the course of the disease in patients with severe asthma, certain inflammatory endotypes, particularly non-eosinophilic asthma, remain deprived of any effective treatment (Ortega et al., 2015; Holgate et al., 2004; Castro et al., 2018).

Bronchial thermoplasty is a therapeutic innovation regarding asthma management. It is an effective endoscopic treatment of moderate to severe asthma, making it possible to reduce smooth muscle mass by administering radiofrequency in the bronchi (Thomson et al., 2012). Although effective, this technique has short-term to long-term side effects and remains a highly invasive procedure.

It is recognized that severe asthma is the clinical form for which bronchial inflammation and remodeling are intertwined. Thus, the development of new treatments acting on these mechanisms is a major public health challenge in that they could help improve the therapeutic management of affected patients and modify disease outcomes (Chanez et al., 2007).

Airway smooth muscle is distributed at the tracheobronchial tree from the trachea to the terminal bronchioles. Cholinergic innervation forms the main bronchoconstrictive control thanks to acetylcholine, which allows the bronchial muscle to contract through its interaction with muscarinic receptors, the subtypes M2 and M3 of which are present on the membrane of human smooth bronchial muscle cells (Kolahian and Gosens, 2012).

Nevertheless, airway smooth muscle contraction can be triggered by a large number of endogenous molecules, such as, inter alia, acetylcholine, histamine, leukotrienes, bradykinin, endothelin-1, angiotensin II, or serotonin or 5-Hydroxytryptamine (5-HT) (Bossé, 2012).

5-HT is one of the most studied central nervous system (CNS) neurotransmitters, known to have very diverse physiological functions, aside from the CNS, such as cytokine production stimulation, vasoconstriction, cell proliferation (fibroblasts, smooth muscle cells, endothelial cells), or inflammatory cell migration (Ménard et al., 2007; Müller et al., 2009; Delaney et al., 2011; Pakala et al., 1997; Boehme et al., 2004; Kushnir-Sukhov et al., 2006; Filip and Bader, 2009). In the lungs, it appears to be involved in chronic inflammatory diseases (Nichols and Nichols, 2008).

5-HT exerts its effects by binding to cellular receptors which are classified into seven distinct families (5-HT1 to 5-HT7) comprising 14 subtypes, according to their structural diversity and their mode of action (Kitson, 2007). The effects of 5-HT on inflammatory cells are mainly provided by one or more of the following receptors: 5-HT1A, 5-HT2A, 5-HT3, 5-HT4 and 5-HT7.

Beyond its functions described above, many studies have demonstrated a role of 5-HT in the pathogenesis of allergy and asthma, in particular in promoting eosinophil recruitment, airway inflammation, exacerbated bronchial reactivity, and remodeling, induced by allergens (Boehme et al., 2004; De Bie et al., 1998; Lima et al., 2007), characteristics of allergic asthma.

It has been demonstrated that elevated circulating serotonin levels were present in patients with asthma and also that these 5-HT levels were correlated positively with patients' clinical status and negatively with lung function, suggesting that 5-HT could play a role in the physiopathology of acute asthma (Lechin et al., 1994; Lechin et al., 1996).

At airway smooth muscles, 5-HT activates both 5-HT_{2A} and 5-HT_{1A} receptors, which are respectively responsible for muscle contraction and relaxation (Cazzola and Matera, 2000). However, it has been demonstrated that stimulating 5-HT_{1A} receptors of respiratory smooth muscle cells made it possible to potentiate bronchoconstriction induced by 5-HT₂ receptor activation (Germonpré et al. 1998).

Although serotonin alone is capable of inducing bronchoconstriction, an interconnection of the serotoninergic and cholinergic systems has been demonstrated. Indeed, presynaptic serotoninergic receptors are also capable of modulating (inhibiting or potentiating) the release of neurotransmitters from airway cholinergic and non-cholinergic nerve fibers (Germonpré et al., 1998; Mendez-Enriquez 2021).

Besides the role of serotonin and its receptors in controlling bronchial smooth muscle fiber contractility, the serotoninergic system also appears to be important in another important component of obstructive disorders: airway remodeling. Serotoninergic receptors, particularly 5-HT2 receptors, play an important role in controlling human bronchial smooth muscle cell proliferation and in pro-fibrotic factor TGF-β1 release (Löfdhal et al. 2018).

It is important to note that some structural alterations of the airways are common characteristics of several respiratory disorders. Indeed, exacerbated bronchial reactivity is also observed in other lung diseases (rhinitis, chronic bronchitis, interstitial lung diseases, cystic fibrosis or in chronic obstructive pulmonary disease (COPD)). COPD is a disease that is characterized by airway obstruction usually caused by chronic bronchitis, emphysema, or both. In COPD, airway obstruction results from chronic and excessive abnormal mucus secretion, inflammation, bronchospasm, and infection. There are very few treatments currently available to relieve COPD symptoms, prevent exacerbations, maintain optimal lung function, and improve patients' day-to-day activities and quality of life.

The link between serotonin and COPD has been clearly established. Indeed, significantly higher circulating serotonin levels have been observed in COPD patients; this appears to be linked to worsening airway obstruction and mortality rate (Pirina et al. 2018; Meier et al. 2017).

It therefore appears to be essential to develop novel pharmacological approaches making it possible to control bronchial contraction, particularly in response to serotonin, and to manage exacerbated airway reactivity in respiratory diseases, in order to limit as much as possible the clinical progression of patients with bronchoconstrictive disorders such as asthma or COPD.

### SUMMARY

The present disclosure is aimed at providing such a treatment. The aim of the present disclosure is that of overcoming the aforementioned drawbacks by providing an effective treatment of inflammatory respiratory diseases and particularly of chronic inflammatory respiratory diseases.

It was discovered by the present inventors that such an aim is achieved by administering at least one phytoecdysone and/or at least one semi-synthetic derivative of 20-hydroxyecdysone, this administration inducing, unexpectedly, bronchodilation and preventing bronchospasm in mammals, in the context of an inflammatory respiratory disease. Chronic administration of at least one phytoecdysone and/or at least one semi-synthetic derivative of 20-hydroxyecdysone induces a reduction in exacerbated bronchial reactivity in a mouse model of severe allergic asthma. This decrease in exacerbated bronchial reactivity is observed both in a bronchoconstriction test induced by methacholine and, even more substantially, when it is induced by serotonin. Remarkably, this decrease in bronchoconstriction in response to serotonin is accompanied by selective reduction in gene expression of a receptor of the serotoninergic system. Indeed, following chronic exposure to phytoecdysone and/or a semi-synthetic derivative of 20-hydroxyecdysone, the gene expression of 5-HT_{1A} serotonin receptor is significantly reduced in mammalian bronchi. Bronchial gene expression of the other 5-HT receptors tested (5-HT_{1B}, 5-HT_{2A}, 5-HT_{2B}, 5-HT₇) and the serotonin transporter (5-HTT) is not significantly affected by chronic administration of phytoecdysone and/or a semi-synthetic derivative of 20-hydroxyecdysone.

Exacerbated bronchial reactivity is associated with extensive lung remodeling. Interestingly, chronic administration of at least one phytoecdysone and/or at least one semi-synthetic derivative of 20-hydroxyecdysone in asthmatic mammals reduces remodeling of the animals' airways. Indeed, in treated asthmatic mice, peribronchial and perivascular inflammation are reduced. Furthermore, the increase in bronchial muscle mass and epithelial barrier dysfunction associated with severe asthma appear to be less substantial in treated animals. This decrease in bronchial smooth muscle cell hyperplasia would explain the decrease in exacerbated bronchial reactivity in asthmatic mice treated with chronic administration of phytoecdysone and/or a semi-synthetic derivative of 20-hydroxyecdysone.

Thus, according to a first aspect, the present disclosure relates to a composition comprising at least one phytoecdysone and/or at least one semi-synthetic derivative of 20-hydroxyecdysone, for its use in the treatment of an inflammatory respiratory disease in mammals.

In the present description, the term “treatment” means obtaining a desired pharmacological and physiological effect. The term “treatment”, as used in the present description, comprises prevention or partial prevention of one or more of the symptoms of the disease and/or partial or total cure of the disease and/or the total or partial disappearance of one or more of its symptoms.

In the present description, “at least one” means a single compound (a phytoecdysone or a semi-synthetic derivative of 20-hydroxyecdysone) or a mixture of several such compounds.

In particular aspects, the disclosure furthermore fulfills the following features, implemented separately or in each of their technically feasible combinations.

A phytoecdysone that can be used according to the disclosure is for example 20-hydroxyecdysone.

Semi-synthetic derivatives of 20-hydroxyecdysone are in particular understood to mean the known compounds covered by patent application WO 2015/177469 wherein their obtention by semisynthesis is described.

The phytoecdysones and the semi-synthetic derivatives of 20-hydroxyecdysone are advantageously purified to pharmaceutical grade.

According to a particular embodiment, the composition according to the present disclosure comprises 20-hydroxyecdysone and/or at least one semi-synthetic derivative of 20-hydroxyecdysone.

The 20-hydroxyecdysone used is preferably in the form of an extract of plants rich in 20-hydroxyecdysone or a composition comprising 20-hydroxyecdysone as the active agent. Extracts of plants rich in 20-hydroxyecdysone are for example extracts of Stemmacantha carthamoides (also called Leuzea carthamoides), Cyanotis arachnoidea, Cyanotis vaga and Pfaffia paniculata.

The extracts obtained are preferably purified to pharmaceutical grade.

In one embodiment, 20-hydroxyecdysone is in the form of an extract of a plant or a plant part, said plant being chosen from plants containing at least 0.5% of 20-hydroxyecdysone by dry weight of said plant, said extract comprising at least 95%, and preferably at least 97%, of 20-hydroxyecdysone. Said extract is preferably purified to pharmaceutical grade.

Said extract is hereinafter called BI0101. It remarkably comprises between 0 and 0.05%, by dry weight of the extract, of impurities, such as minor compounds, capable of affecting the safety, availability or efficacy of a pharmaceutical application of said extract.

According to an aspect of the disclosure, the impurities are compounds with 19 or 21 carbon atoms, such as Rubrosterone, Dihydrorubrosterone or Poststerone.

The plant from which BI0101 is produced is preferably chosen from Stemmacantha carthamoides (also called Leuzea carthamoides), Cyanotis arachnoidea, Cyanotis vaga and Pfaffia paniculata.

According to a particular embodiment, the inflammatory respiratory disease is a chronic inflammatory respiratory disease.

According to a preferred embodiment, the composition according to the present disclosure is used as a bronchodilator in the treatment of the inflammatory respiratory disease in mammals.

The composition according to the present disclosure is intended to be used to induce bronchodilation in a patient with an inflammatory respiratory disease. Thus, in particular aspects, the disclosure relates to a composition comprising at least one phytoecdysone and/or at least one semi-synthetic derivative of 20-hydroxyecdysone, for its use in inducing bronchodilation in the treatment of a mammal having an inflammatory respiratory disease.

In the present description, the term “bronchodilator” refers to any compound which dilates the bronchi and bronchioles and, in doing so, increases airflow to the lungs. They are useful in bronchoconstrictive disorders such as asthma, which involve airflow obstruction and bronchospasm.

In the present description, the term “bronchodilation” refers to the expansion of bronchial airways to treat or prevent a bronchoconstrictive disorder.

In the present description, the term “bronchoconstrictive disorder” refers to any disorder or disease linked to reduction of the inner diameter of the bronchial tract, for example, a bronchus or bronchi, including, but not limited to, asthma, chronic obstructive pulmonary disease (COPD) and cystic fibrosis.

According to a particular embodiment, the disclosure relates to a composition comprising at least one phytoecdysone and/or at least one semi-synthetic derivative of 20-hydroxyecdysone, for use in the treatment of asthma, chronic obstructive pulmonary disease or cystic fibrosis.

The composition according to the present disclosure is furthermore intended to be used in the prevention or reduction of exacerbated bronchial reactivity in a patient having an inflammatory respiratory disease. Thus, in particular aspects, the disclosure relates to a composition comprising at least one phytoecdysone and/or at least one semi-synthetic derivative of 20-hydroxyecdysone, for its use in the prevention or reduction of exacerbated bronchial reactivity in the treatment of a mammal having an inflammatory respiratory disease.

According to the disclosure, exacerbated bronchial reactivity refers to an airway abnormality which consists of an exaggerated airway contraction response to many environmental triggers, such as allergens or exercise, but is not limited thereto. Exacerbated bronchial reactivity may be a functional impairment of the respiratory system caused by airway inflammation or remodeling. Exacerbated bronchial reactivity may be caused by collagen deposition, bronchospasm, airway smooth muscle hypertrophy, airway smooth muscle contraction, mucus secretion, cell deposits, epithelial destruction, epithelial permeability impairment, smooth muscle function or sensitivity impairment, lung parenchymal abnormalities and/or infiltrative diseases in and around the airways.

Many of these causal factors can be associated with inflammation. The exacerbated bronchial reactivity targeted by treatment with the composition according to the present disclosure is associated with airway inflammation (for example, inflammatory cytokine production).

In a particular embodiment, the disclosure relates to the composition comprising at least one phytoecdysone and/or at least one semi-synthetic derivative of 20-hydroxyecdysone for its use in the treatment of respiratory function impairment linked to the serotoninergic pathway in the mammal having an inflammatory respiratory disease.

In a particular embodiment, the disclosure relates to the composition comprising at least one phytoecdysone and/or at least one semi-synthetic derivative of 20-hydroxyecdysone for its use in the treatment of respiratory function impairment linked to the serotoninergic pathway 5-HT_{1A} receptor in the mammal having an inflammatory respiratory disease.

In a particular embodiment, the phytoecdysones are administered at a dose of between 3 and 15 milligrams per kilogram per day in humans. Phytoecdysone here means both phytoecdysones in general, in particular 20-hydroxyecdysone and preferably in the form of an extract, and the semi-synthetic derivatives of 20-hydroxyecdysone.

Preferably, the phytoecdysones are administered at a dose of 200 to 1000 mg/day, in one or more times, in adult humans, and a dose of 5 to 350 mg/day, in one or more times, in human children or infants. Phytoecdysone here means both phytoecdysones in general, in particular 20-hydroxyecdysone and preferably in the form of an extract, and the semi-synthetic derivatives of 20-hydroxyecdysone.

In particular aspects of the present disclosure, said at least one semi-synthetic derivative of 20-hydroxyecdysone is chosen from:
a compound of general formula (I):

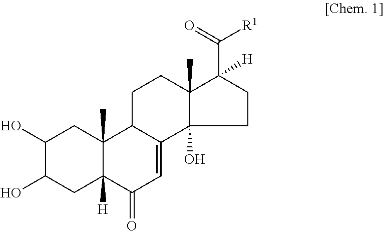

wherein:
R¹ is chosen from: a (C₁-C₆)W(C₁-C₆) group; a (C₁-C₆)W(C₁-C₆)W(C₁-C₆) group; a (C₁-C₆)W(C₁-C₆)CO₂(C₁-C₆) group; a (C₁-C₆)A group, A representing a heterocycle optionally substituted by an OH, OMe, (C₁-C₆), N(C₁-C₆), CO₂(C₁-C₆) type group; a CH₂Br group;
W being a heteroatom chosen from N, O and S, preferably 0 and even more preferably S; and,
a compound having formula (II):

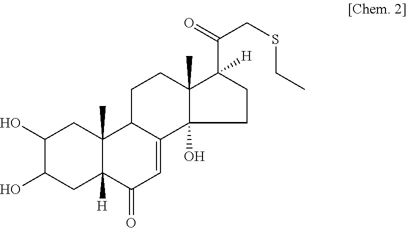 [SMILES: CCSCC(=O)[C@H]1CC[C@@]2(O)C3=CC(=O)[C@@H]4CC(O)C(O)C[C@]4(C)C3CC[C@]12C]

Within the scope of the present disclosure, “(C₁-C₆)” means any linear or branched alkyl group of 1 to 6 carbon atoms, in particular, the methyl, ethyl, n-propyl, iso-propyl groups, n-butyl, iso-butyl, sec-butyl, t-butyl, n-pentyl, n-hexyl groups. Advantageously, it consists of a methyl, ethyl, isopropyl or t-butyl group, in particular a methyl or ethyl group, more particularly a methyl group.

Within the scope of the present disclosure, heterocycle preferably means a ring comprising 5 or 6 atoms including one or two heteroatoms (0, S or N), the remaining atoms being carbon atoms.

In a preferred aspect of the present disclosure, in general formula (I):
R¹ is chosen from: a (C₁-C₆)W(C₁-C₆) group; a (C₁-C₆)W(C₁-C₆)W(C₁-C₆) group; a (C₁-C₆)W(C₁-C₆)CO₂(C₁-C₆) group; a (C₁-C₆)A group, A representing a heterocycle optionally substituted by an OH, OMe, (C₁-C₆), N(C₁-C₆), CO₂(C₁-C₆) type group; W being a heteroatom chosen from N, O and S, preferably O and more preferably S.

In particular aspects of the present disclosure said at least one semi-synthetic derivative of 20-hydroxyecdysone is a compound chosen from the following compounds:
No. 1: (2S,3R,5R,10R,13R,14S,17S)-2,3,14-trihydroxy-10.13-dimethyl-17-(2-morpholinoacetyl)-2,3,4,5,9,11,12,15,16,17-decahydro-1H-cyclopenta[a]phenanthren-6-one,
No. 2: (2S,3R,5R,10R,13R,14S,17S)-2,3,14-trihydroxy-17-[2-(3-hydroxypyrrolidin-1-yl)acetyl]-10,13-dimethyl-2,3,4,5,9,11,12,15,16,17-decahydro-1H-cyclopenta[a]phenanthren-6-one;
No. 3: (2S,3R,5R,10R,13R,14S,17S)-2,3,14-trihydroxy-17-[2-(4-hydroxy-1-piperidyl)acetyl]-10,13-dimethyl-2,3,4,5,9,11,12,15,16,17-decahydro-1H-cyclopenta[a]phenanthren-6-one;
No. 4: (2S,3R,5R,10R,13R,14S,17S)-2,3,14-trihydroxy-17-[2-[4-(2-hydroxyethyl)-1-piperidyl]acetyl]-10,13-dimethyl-2,3,4,5,9,11,12,15,16,17-decahydro-1H-cyclopenta[a]phenanthren-6-one;
No. 5: (2S,3R,5R,10R,13R,14S,17S)-17-[2-(3-dimethylaminopropyl(methyl)amino)acetyl]-2,3,14-trihydroxy-10,13-dimethyl-2,3,4,5,9,11,12,15,16,17-decahydro-1H-cyclopenta[a]phenanthren-6-one;
No. 6: 2-[2-oxo-2-[(2S,3R,5R,10R,13R,14S,17S)-2,3,14-trihydroxy-10,13-dimethyl-6-oxo-2,3,4,5,9,11,12,15,16,17-decahydro-1H-cyclopenta[a]phenanthren-17-yl]ethyl]ethyl sulfanylacetate;
No. 7: (2S,3R,5R,10R,13R,14S,17S)-17-(2-ethylsulfanylacetyl)-2,3,14-trihydroxy-10.13-dimethyl-2,3,4,5,9,11,12,15,16,17-decahydro-1H-cyclopenta[a]phenanthren-6-one;
No. 8: (2S,3R,5R,10R,13R,14S,17S)-2,3,14-trihydroxy-17-[2-(2-hydroxyethylsulfanyl)acetyl]-10,13-dimethyl-2,3,4,5,9,11,12,15,16,17-decahydro-1H cyclopenta[a]phenanthren-6-one.

In some aspects, the composition according to the present disclosure is a pharmaceutical composition containing, as an active ingredient, at least one phytoecdysone and/or at least one semi-synthetic derivative of 20-hydroxyecdysone, in a pharmaceutically acceptable vehicle.

In the present description, “pharmaceutically acceptable vehicle” means any vehicle useful for the preparation of a pharmaceutical composition and which is generally safe, non-toxic and neither biologically nor otherwise undesirable for the subject to be treated, in particular for mammals and particularly humans.

The vehicle of the pharmaceutical composition according to the disclosure can be solid, semi-solid or liquid. It can consist of a diluent, an adjuvant, or any other vehicle conventional per se for composing pharmaceutical compositions.

The pharmaceutical composition according to the disclosure can be in any dosage form, in particular in a form adapted for administration by the parenteral, intranasal, rectal, pulmonary, intrathecal, systemic or topical route. Preferably, it is in a form suitable for oral administration. By way of non-limiting examples of such dosage forms according to the disclosure, mention may be made of granule, powder, tablet, capsule, pill, syrup, solution or oral suspension forms, etc.

The administration of the compound used according to the disclosure to the subject to be treated can be carried out by any conventional route per se, in particular by the parenteral route, for example by the subcutaneous, subdural, intravenous, intramuscular, intrathecal, intraperitoneal, intracerebral, intra-arterial or intralesional route; by the intranasal route; by the rectal route; by the pulmonary route, for example by aerosol or inhalation; or even by the topical route. It is preferably carried out per os.

Any pharmaceutically acceptable conventional salt of the compound of general formula (I) can be implemented according to the disclosure. By way of examples, mention may be made of chlorides, bromides, formates, acetates, etc.

In the present description, “pharmaceutically acceptable salt” means, in a conventional manner per se, any salt of the compound of general formula (I) comprising, as a counterion, a substance producing no adverse, allergic or otherwise undesirable reaction when administered to a subject, in particular to a mammal.

The cosmetic composition according to the disclosure, in its desired form, can be prepared by any conventional method per se for the preparation of pharmaceutical compositions.

The composition according to the disclosure can contain one or more excipients/additives conventional per se for composing pharmaceutical compositions, for example chosen from preservatives, sweetening agents, flavoring agents, fillers, disintegrating agents, wetting agents, emulsifiers, surfactants, dispersing agents, lubricants, stabilizers, buffers, antibacterials, antifungals, etc., or any of the mixtures thereof; and/or any compound allowing rapid, prolonged or delayed, and/or targeted, release of the active ingredient after its administration to the subject.

The composition according to the disclosure can further contain one or more active ingredients other than phytoecdysones or semi-synthetic derivatives of 20-hydroxyecdysone, where these active ingredients may or may not act synergistically with said phytoecdysones or said semi-synthetic derivatives of 20-hydroxyecdysone.

The pharmaceutical composition according to the disclosure is preferably formulated in unit dose form.

The disclosure is also expressed in terms of a method for treating an inflammatory respiratory disease, comprising administering to a subject in need thereof a therapeutically effective amount of a composition comprising at least one phytoecdysone and/or at least one semi-synthetic derivative of 20-hydroxyecdysone. This method may fulfill one or more of the features described above with reference to the therapeutic use of the composition comprising at least one phytoecdysone and/or at least one semi-synthetic derivative of 20-hydroxyecdysone as a drug.

In the present description, “a subject in need thereof” means a subject affected by or likely to be affected by an inflammatory respiratory disease. This subject can in particular be a mammal, and in particular a human.

In the present description, “therapeutically effective amount” means the amount of the administered composition which, when administered to a subject to treat the disease, is sufficient to provide such a treatment of the disease. The therapeutically effective amount of the composition used according to the disclosure is dependent on several factors, such as the disease and its severity, age, weight, etc., of the subject to be treated, the particular compound(s) of the composition used, the route and form of administration, etc. The therapeutically effective amount of the composition used according to the disclosure will be determined by the physician for each individual case. For example, the composition may be administered to the subject in need thereof once, twice, or three times a day, over a long period, at regular intervals, or in a targeted manner.

The disclosure is also expressed in terms of use of a composition comprising at least one phytoecdysone and/or at least one semi-synthetic derivative of 20-hydroxyecdysone for the manufacture of a medicinal product for the treatment of an inflammatory respiratory disease. This use may contain one or more of the features described above, implemented separately or in each of their technically feasible combinations.

### BRIEF DESCRIPTION OF THE FIGURES

The disclosure will be better understood upon reading the following description, given as a non-limiting example, and made with reference to the figures which represent:

FIG. 1 shows the timeline of the HDM (House Dust Mite) study wherein allergic asthma model mice are treated with BI0101;

FIG. 2 illustrates the bronchial ring contraction response curve in healthy (control) or asthmatic (asthma) mice and also the effects of the BI0101 molecule (50 mg/kg/day) on bronchial ring contraction in control (control+BI0101) and asthmatic (Asthma+BIO101) mice. Bronchoconstriction was induced by stimulation by cumulative doses of (A) methacholine (10⁻⁸M to 10⁻⁴M) or (B) 5-HT (10⁻⁸M to 10⁻⁴M). The values are shown as means±SEM where p<0.001 ###control vs Asthma, and Asthma vs Asthma+BI0101, p<0.001****.

FIG. 3 shows a histogram illustrating the gene expression of 5-HT receptors and their transporter in the bronchi of healthy mice (control), control mice treated with BI0101 (control+BI0101), asthmatic mice (asthma) and asthmatic mice treated with BI0101 (asthma+BIO101). The values are shown as means±SEM. The values are shown as means ±SEM where p<0.05.

FIG. 4 shows the plots representative of real-time recordings of the bronchial ring response in of healthy mice (A: control) and severe allergic asthma model mice (B: asthma) following sequential administration of serotonin before then after incubation with WAY-101405, a specific 5-HT1A receptor antagonist. Scale bar: 1 minute for the x-axis and 1 mN for the y-axis.

FIG. 5 shows images representing lung sections stained with hematoxylin and eosin from healthy mice (A: control) and mice treated with BI0101 (B: control+BIO101), asthmatic mice (C: asthma) and asthmatic mice treated with BI0101 (D: asthma+BIO101). The scale bar represents 100 μm.

FIG. 6 shows high-magnification images, representing lung sections stained with hematoxylin and eosin from healthy mice (A: control) and mice treated with BI0101 (B: control+B10101), asthmatic mice (C: asthma) and asthmatic mice treated with BI0101 (D: asthma+BIO101). The scale bar represents 100 μm.

FIG. 7 shows the bronchoalveolar fluid cellularity values for control mice (n=9), control mice treated with BI0101 (n=9), asthmatic mice (n=10) and asthmatic mice treated with BIO101 (n=11). The cellularity is calculated by dividing the total number of cells in the sample by the volume of bronchoalveolar fluid collected.

FIG. 8 shows the Penh value curve of control mice (n=10, white squares), control mice treated with BI0101 (n=10; white circles), asthmatic mice (n=10; black squares) and asthmatic mice treated with BI0101 (n=11; black triangles), measured by plethysmography, in response to increasing doses of methacholine where *p<0.05 and ****p<0.0001.

### DETAILED DESCRIPTION

In the present description, n corresponds to the sample size and p corresponds to the “p-value” used to quantify the statistical significance of a result.

A Mann-Whitney test was performed for comparisons between two groups. A two-way ANOVA test was used for multiple comparisons of bronchial contractility studies. The data analysis was carried out using GraphPad Prism 9 software (GraphPad Software, Inc., La Jolla, CA, USA). The disclosure will be described hereinafter in the particular context of one of its preferred, non-limiting, fields of application.

### 1. Study Description:

Seven-week old mice were sensitized by cutaneous route to house dust mite (HDM) extracts on DO, 7, 14 and 21. After this skin sensitization phase to the allergen, the mice receive the allergen by the nasal route (challenge phase) on D27, 28, 29, 34, 35 and 36 to induce bronchial exacerbation. This severe allergic asthma model has been characterized as mimicking human pathology (Dilasser et al. Thorax 2021).

BIG101 (batch RL11T1204A0) was administered to the mice in drinking water 7 days a week, at a dose of 50 mg/kg/day. The drinking water containing BI0101 was replaced twice weekly until the animals were sacrificed.

### 2. Biological Activity of BI0101 in a Mouse Model of Severe Allergic Asthma

a. Analysis of the Effects of BI0101 Treatment on Bronchoconstriction

The evaluation of the effect of chronic administration of BI0101 treatment on ex vivo murine bronchial ring contraction was carried out in an isolated organ chamber (isometric tension measurement, Mulvany myograph, DMT, Hinnerup, Denmark) (André-Grégoire et al., 2018).

The bronchi are collected, cleaned, cut into rings and set up in the Mulvany myograph. The rings are pre-stretched and then stimulated with 60 mM KCl until the contractile response amplitude has stabilized (2 or 3 stimulations) before the start of each experimental protocol.

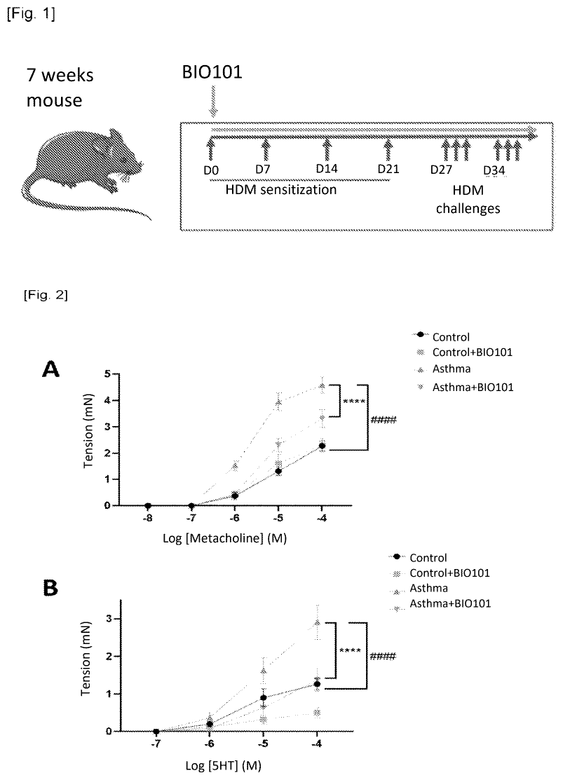

On D38 after the start of the protocol (FIG. 1), the bronchi of healthy non-asthmatic mice (control; n≥8), healthy non-asthmatic mice treated with BI0101 (control+BIO101; n=8), asthmatic mice (Asthma; n≥8) and asthmatic mice treated with BIG101 (Asthma+BIO101; n≥8) were collected. Bronchoconstriction was induced by stimulation by cumulative doses of metacholine (10⁻⁷M to 10⁻⁴M) or serotonin (10⁻⁷M to 10⁴M).

The experiments were carried out on 2 bronchial rings from each mouse. The drug BI0101 was added at a concentration of 10⁻⁵M in the isolated organ chambers containing the treated mouse bronchi. This made it possible to maintain tissue treatment throughout the experiment. It should be noted that acute administration of BIG101 in the vessel containing the bronchial ring does not modify the contractile response to methacholine (0.41±0.25 mN) relative to the internal control without BI0101 (0.37±0.22 mN; p=ns) or to serotonin (0.29±0.14 mN) relative to the internal control without BI0101 (0.25±0.12 mN; p=ns) (previous results obtained, not shown here).

As expected, in asthmatic mice, methacholine- and serotonin-induced bronchoconstrictions are significantly higher than those of healthy mice (p<0.0001 for each of the conditions), which confirms that sensitization to house dust mites induced exacerbated bronchial reactivity (FIG. 2).

In asthmatic mice, chronic treatment with BI0101 significantly decreases methacholine-induced bronchoconstriction in asthmatic mice (Asthma vs. Asthma+BIO101; p<0.0001). Nevertheless, treatment does not prevent exacerbated bronchial airway reactivity and does not achieve a comparable level of bronchoconstriction to that of healthy mice (Asthma+BIO101 vs Control; p=0.0025).

Similarly, chronic treatment with BI0101 significantly reduces serotonin-induced bronchoconstriction in asthmatic mice (Asthma vs. Asthma+BSO101; p<0.0001). Remarkably, it is observed that chronic treatment with BI0101 induces comparable serotonin-induced bronchoconstriction to that observed with bronchi of healthy mice (Asthma+BIO101 vs Control; p=0.9997), demonstrating that the treatment completely prevents exacerbated bronchial airway reactivity.

b. Analysis of the Effect of Chronic BI0101 Treatment on Gene Expression of 5-HT Receptors and Transporter in Bronchi

On D38, the bronchi of healthy non-asthmatic mice (control; n=10), non-asthmatic control mice treated with BI0101 (controls+BIO101; n=10), asthmatic mice (Asthma; n=10) and asthmatic mice treated with BI0101 (Asthma+BIO101; n=10) were collected. After RNA extraction from these tissues (Trizol©, ThermoFisher cat. 15596018), an analysis of serotonin receptor (5-HT1A, 5-HT1B, 5-HT2A, 5-HT2B and 5-HT7) and serotonin transporter (5HTT) expression was evaluated with real-time PCR (TaqMan™, Universal PCR MasterMix, ThermoFisher cat. 4304437). The list of ThermoFisher Scientific commercial test references used for real-time PCR to obtain the results for each of the genes is given in Table 1 below:

TABLE 1

| Gene symbol | Test Reference |
|---|---|
| Htt | Mm01213820_m1 |
| Htr7 | Mm00434133_m1 |
| Htr2a | Mm00555764_m1 |
| Htr2b | Mm00434123_m1 |
| Htr1a | Mm00434106_s1 |
| Htr1b | Mm00439377_s1 |
| Gapdh | Mm99999915_g1 |
| Hprt | Mm03024075_m1 |

Chronic treatment of mice with BI0101 induces a significant and selective decrease in 5-HT1A receptor gene expression in bronchi (FIG. 3). Indeed, 5-HT1A receptor gene expression decreases by −49%±12% (p<0.05) in healthy mice treated with BI0101 (control+BIO101) and even more substantially in asthmatic mice treated with BI0101 (asthma+BIO101) (−69±3%; p<0.05).

Treatment with BI0101 does not induce any modification of other serotonin receptors or transporter, whether in mice are healthy or asthmatic (5-HT1B, 5-HT2A, 5-HT2B and 5-HT7 and 5-HTT).

c. Confirmation of the Involvement of 5-HTIA Receptor in Serotonin-Induced Bronchoconstriction.

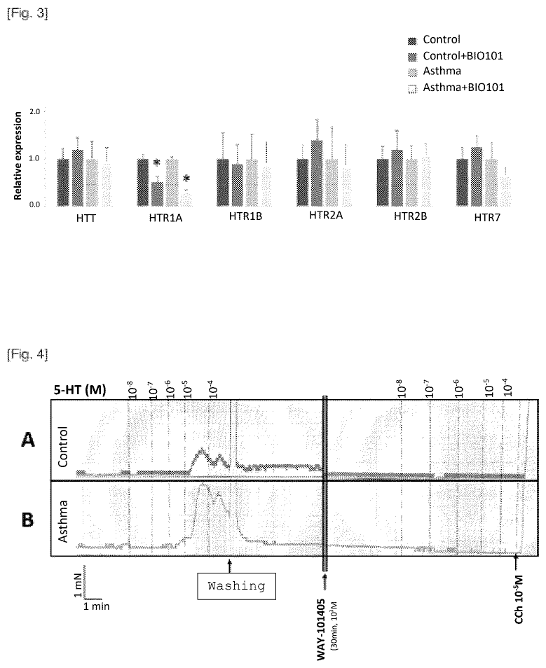

Real-time recordings of the response of bronchi of control and asthmatic mice following sequential administration of serotonin at different concentrations (10⁻⁸M to 10⁴M) were carried out. Following this recording, the bronchial rings were incubated for 30 minutes with WAY-101405, a selective 5-HT1A receptor antagonist (FIG. 4).

As expected, serotonin induces bronchoconstriction in bronchial rings of healthy (control) and asthmatic mice. Bronchoconstriction of bronchi of asthmatic mice (FIG. 4, B) being more substantial than that of healthy mice (FIG. 4, A). Serotonin-induced bronchoconstriction is totally inhibited by the 5-HT1A receptor antagonist (WAY-101405) in control and asthmatic mice (FIGS. 4, A and B).

This result suggests that serotonin-dependent bronchoconstriction is essentially mediated by 5-HT1A receptor activation.

All of these results strongly suggest that the selective decrease in 5-HT1A receptor expression induced by chronic BI0101 treatment could significantly contribute to the prevention of exacerbated bronchial airway reactivity in an inflammatory disease context.

d. Histopathological Analysis of the Effects of Chronic BI0101 Treatment in the Lungs

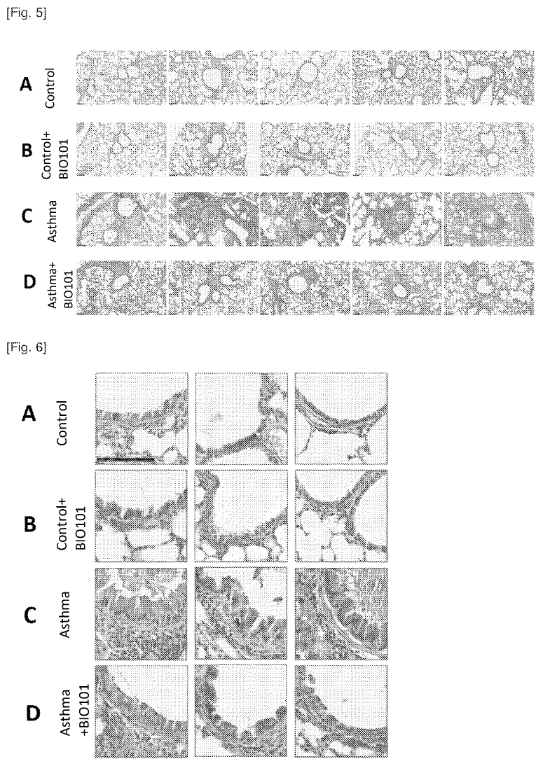

The histological analysis of sections of lungs stained with hematoxylin and eosin shows that exacerbated bronchial airway reactivity of mice sensitized to house dust mites (asthma, FIG. 5 in C and FIG. 6 in C) is associated with extensive lung remodeling characterized by mucus production, substantial cellular infiltration of the airways, epithelial cell hypertrophy, and also smooth muscle cell hyperplasia, relative to the lungs of healthy mice (FIG. 5 in A and FIG. 6 in A).

In healthy mice (control), BIG101 treatment has no effect on lung histology ((FIG. 5 in B and FIG. 6 in B). On the other hand, in asthmatic mice, BI0101 treatment reduces peribronchial and perivascular inflammation ((FIG. 5 in D and FIG. 6 in D). Furthermore, the increase in bronchial muscle mass and epithelial barrier dysfunction associated with severe asthma appear to be less substantial in animals having received chronic BI0101 treatment.

### E. Evaluation of Bronchoalveolar Fluid Cellularity

On D38, sterile PBS (between 200 μL and 1 mL) was administered by the intratracheal route to the mice using a catheter. The bronchoalveolar fluid (BALF) cells and supernatant are retrieved and separated by centrifugation. The total number of cells was counted on Kova slides by optical microscopy. The cellularity of each sample was evaluated by calculating the ratio of the total number of cells counted, divided by the volume of bronchoalveolar fluid. The cellularity represents the infiltrate of immune cells (macrophages, lymphocytes, eosinophils and neutrophils).

There is no difference in the cell content of BALFs from control mice treated with BI0101 and of untreated control mice (260±64 and 244±46 respectively, p=ns). As expected, the cellularity of BALFs from asthmatic mice increases substantially (721±248; +177.3%) relative to the control group (FIG. 7). Interestingly, although not significantly, treatment of asthmatic mice with BI0101 reduces the inflammation present in BALFs (−39.8%; p=ns).

This result is consistent with the histological analysis of lung sections wherein it is observed that treatment with BI0101 induces a decrease in airway inflammation and remodeling.

f. Analysis of Respiratory Function by Plethysmography

In order to study whether the reduction of inflammation in BALF could induce functional improvement after severe asthma induction, respiratory function was evaluated by plethysmography. This technique makes it possible to carry out a longitudinal study of respiratory capacity and also expiratory pause time (Penh) non-invasively. Breathing-induced flow and pressure changes are measured before and after exposure to nebulization with increasing doses of NaCl followed by methacholine. The total recording for one mouse lasts 45 minutes. The alert, unrestrained mouse is placed in an isolated plethysmography chamber and a single dose of methacholine is nebulized to the mouse for one minute, and then for 5 minutes when the data recording is performed. After these 5 minutes, a new dose of methacholine is nebulized, and so on. Over the 45 minutes, the mouse undergoes a total of 5 nebulizations with increasing doses of methacholine diluted with NaCl (0, 5, 10, 20, 40 mg/ml).

The pressure variations measured relative to a reference chamber make it possible to define various respiratory parameters such as inspiratory and expiratory pressure peaks and times, and also a unitless parameter referred to as Penh (enhanced Pause) which makes it possible to evaluate bronchoconstriction. Indeed, the Penh value, calculated from the pressure signal in the chamber (Pb), is an important index to obtain, because changes in the Penh value evolve in parallel with those of respiratory resistance and therefore it represents a predictive parameter of changes in resistive properties of the respiratory system (Hamelmann et al., 1997; Bergren, 2001; Onclinx et al., 2003). The following values were calculated from the filtered Pb: the maximum change in Pb during expiration (PEP for “Peak Expiratory Pressure”), the maximum change in Pb during inspiration (PIP for “Peak Inspiratory Pressure”), and the time interval (TR). The Penh value was then calculated as follows:

$$Penh = ( PIP / PEP ) \times Pause where \qquad [ Math . 1 ]$$

$$Pause = ( TE - TR ) / TE \qquad [ Math . 2 ]$$

where TE is the expiratory time (Adler et al., 2004).

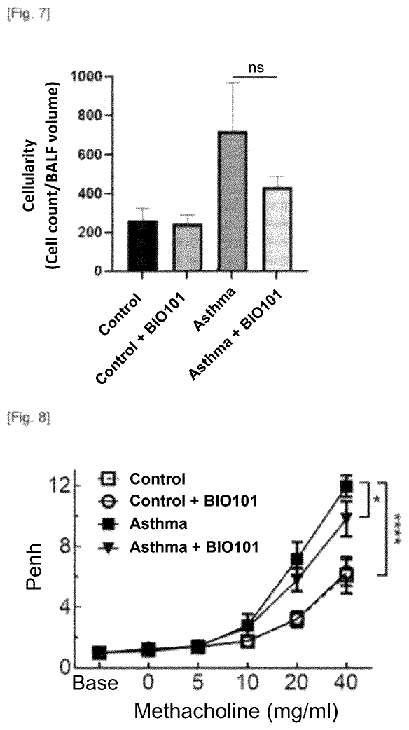

The increase in lung resistance induced by methacholine in aerosol form (Penh) in control mice and control mice treated with BI0101 is comparable. In this study, BI0101 does not appear to have an effect on the respiratory function of healthy animals (FIG. 8).

As expected and described in the literature (Dijoux et al., 2023), it is observed that the Penh value increases significantly in the asthmatic mice group (11.97±0.7) relative to healthy control mice (6.10±1.21; p<0.0001), at 40 mg/ml of methacholine.

Treatment with BI0101 significantly reduces methacholine-induced exacerbated bronchoconstriction. Indeed, administering BIG101 makes it possible to reduce the Penh value by 18.1% (11.97±0.7 in the asthmatic mice group versus 9.81±1.1 in the asthmatic mice group treated with BI0101; p<0.05).

### CONCLUSION

These results demonstrate the benefit of using BI0101 treatment in the context of inflammatory respiratory disease in order to reduce exacerbated bronchial reactivity. Indeed, BI0101 shows substantial beneficial effects in a mouse model of severe allergic asthma, in particular in terms of methacholine- and serotonin-induced bronchoconstriction. Chronic administration of BI0101 induces a significant decrease in 5-HT1A receptor in the bronchi and also a decrease in airway inflammation and remodeling. Treatment with BI0101 (50 mg/kg per day in drinking water) improves respiratory function in a mouse model of severe allergic asthma. This improvement in respiratory function may be linked to the decrease in cellularity detected in BALFs from mice having received BI0101. Indeed, the decrease in inflammatory infiltrate is a key parameter in the clinical improvement of patients suffering from inflammatory respiratory diseases, such as asthma.

### BIBLIOGRAPHICAL REFERENCES

Adler A, Cieslewicz G, Irvin C G. Unrestrained plethysmography is an unreliable measure of airway responsiveness in BALB/c and C57BL/6 mice. J. Appl. Physiol. (1985). (2004); 97(1):286-92.
André-Grégoire G, Dilasser F, Chesné J, Braza F, Magnan A, Loirand G, Sauzeau V. Targeting of Rad prevents bronchoconstriction and airway hyperresponsiveness. J Allergy Clin Immunol. 2018 September; 142(3):824-833.e3. doi: 10.1016/j.jaci.2017.09.049. Epub 2017 Nov. 16. PMID: 29155102.
Bergren D R. Chronic tobacco smoke exposure increases airway sensitivity to capsaicin in awake guinea pigs. j. Appl. Physiol. (2001); 90:695-704.
Bloom Cl, Saglani S, Feary J, Jarvis D, Quint J K. Changing prevalence of current asthma and inhaled corticosteroid treatment in the UK: population-based cohort 2006-2016. Eur Respir J 2019 April; 53(4).
Boehme S A, Lio F M, Sikora L, Pandit T S, Lavrador K, Rao S P, Sriramarao P. Cutting edge: serotonin is a chemotactic factor for eosinophils and functions additively with eotaxin. J Immunol. 2004 Sep. 15; 173(6):3599-603. doi: 10.4049/jimmunol.173.6.3599. PMID: 15356103.
Bossé Y. Asthmatic airway hyperresponsiveness: the ants in the tree. Trends Mol Med. 2012 November; 18(11):627.33. doi: 10.1016/j.molmed.2012.09.002. Epub 2012 Oct. 10. PMID: 23062358.
Boulet L P. Airway remodeling in asthma: update on mechanisms and therapeutic approaches. Curr Opin Pulm Med. 2018 January; 24(1):56-62. doi: 10.1097/MCP.0000000000000441. PMID: 29076828.
Castro M, Corren J, Pavord I D, Maspero J, Wenzel S, Rabe K F, Busse W W, Ford L, Sher L, FitzGerald J M, Katelaris C, Tohda Y, Zhang B, Staudinger H, Pirozzi G, Amin N, Ruddy M, Akinlade B, Khan A, Chao J, Martincova R, Graham N M H, Hamilton J D, Swanson B N, Stahl N, Yancopoulos G D, Teper A. Dupilumab Efficacy and Safety in Moderate-to-Severe Uncontrolled Asthma. N Engl J Med. 2018 Jun. 28; 378(26):2486-2496. doi: 10.1056/NEJMoa1804092. Epub 2018 May 21. PMID: 29782217.
Cazzola I, Matera M G. 5-HT modifiers as a potential treatment of asthma. Trends Pharmacol Sci. 2000 January; 21 (1):13-6. doi: 10.1016/s0165-6147(99)01408-x. PMID: 10637649.
Chanez P, Wenzel S E, Anderson G P, Anto J M, Bel E H, Boulet L P, Brightling C E, Busse W W, Castro M, Dahlen B, Dahlen S E, Fabbri L M, Holgate S T, Humbert M, Gaga M, Joos G F, Levy B, Rabe K F, Sterk P J, Wilson S J, Vachier I. Severe asthma in adults: what are the important questions?J Allergy Clin Immunol. 2007 June; 119(6):1337-48. doi: 10.1016/j.jaci.2006.11.702. Epub 2007 Apr. 9. PMID: 17416409.
De Bie J J, Henricks P A, Cruikshank W W, Hofman G, Jonker E H, Nijkamp F P, Van Oosterhout A J. Modulation of airway hyperresponsiveness and eosinophilia by selective histamine and 5-HT receptor antagonists in a mouse model of allergic asthma. Br J Pharmacol. 1998 July; 124(5):857-64. doi: 10.1038/sj.bjp.0701901. PMID: 9692769; PMCID: PMC1565459.
Delaney C, Gein J, Grover T R, Roe G, Abman S H. Pulmonary vascular effects of serotonin and selective serotonin reuptake inhibitors in the late-gestational ovine fetus. Am J Physiol Lung Cell Mol Physiol. 2011 December; 301(6):L937-44. doi: 10.1152/ajplung.00198.2011. Epub 2011 Sep. 9. PMID: 21908589; PMCID: PMC3233823.
Dijoux E, Klein M, Misme-Aucouturier B, Cheminant M A, de Carvalho M, Collin L, Hassoun D, Delage E, Gourdel M, Loirand G, Sauzeau V, Magnan A, Bouchaud G. Allergic Sensitization Driving Immune Phenotyping and Disease Severity in a Mouse Model of Asthma. Allergy Asthma Immunol Res. 2023 March; 15(2):246-261.
Dilasser F, Rose L, Hassoun D, Klein M, Rousselle M, Brosseau C, Guignabert C, Taillé C, Dombret M C, Di Candia L, Heddebaut N, Bouchaud G, Pretolani M, Magnan A, Loirand G, Sauzeau V. Essential role of smooth muscle Rac1 in severe asthma-associated airway remodelling. Thorax. 2021 April; 76(4):326-334. doi: 10.1136/thoraxjnl-2020-216271. Epub 2021 Feb. 4. PMID: 33542087; PMCID: PMC7982925.
Filip M, Bader M. Overview on 5-HT receptors and their role in physiology and pathology of the central nervous system. Pharmacol Rep. 2009 September-October; 61(5):761-77. doi: 10.1016/s1734-1140(09)70132-x. PMID: 19903999.
Germonpré PR, Joos G F, Pauwels R A. Modulation by 5-HT1A receptors of the 5-HT2 receptor-mediated tachykinin-induced contraction of the rat trachea in vitro. Br J Pharmacol. 1998 April; 123(8):1571-8. doi: 10.1038/sj.bjp.0701771. PMID: 9605563; PMCID: PMC1565327.
Global burden of 369 diseases and injuries in 204 countries and territories, 1990-2019: a systematic analysis for the Global Burden of Disease Study 2019. Lancet. 2020; 396(10258): 1204-22.
Goldsmith A M, Hershenson M B, Wolbert M P, Bentley J K. Regulation of airway smooth muscle alpha-actin expression by glucocorticoids. Am J Physiol Lung Cell Mol Physiol. 2007 January; 292(1):L99-L106. doi: 10.1152/ajplung.00269.2006. Epub 2006 Sep. 15. PMID: 16980374.
Goto K, Chiba Y, Sakai H, Misawa M. Glucocorticoids inhibited airway hyperresponsiveness through downregulation of CPI-17 in bronchial smooth muscle. Eur J Pharmacol. 2008 Sep. 4; 591 (1-3):231-6. doi: 10.1016/j.ejphar.2008.06.021. Epub 2008 Jun. 11. PMID: 18577381.
Habib N, Pasha M A, Tang D D. Current Understanding of Asthma Pathogenesis and Biomarkers. Cells. 2022 Sep. 5; 11(17):2764. doi: 10.3390/cells11172764. MID: 36078171; PMCID:PMC9454904.
Hamelmann E, Schwarze J, Takeda K, Oshiba A, Larsen G L, Irvin C G, Gelfand E W. Noninvasive measurement of airway responsiveness in allergic mice using barometric plethysmography. Am. J. Respir. Crit. Care Med. (1997); 156:766-75.
Holgate S T, Chuchalin A G, Hébert J, Lötvall J, Persson G B, Chung K F, Bousquet J, Kerstjens H A, Fox H, Thirlwell J, Cioppa G D; Omalizumab 011 International Study Group. Efficacy and safety of a recombinant anti-immunoglobulin E antibody (omalizumab) in severe allergic asthma. Clin Exp Allergy. 2004 April; 34(4):632-8. doi: 10.1111/j.1365-2222.2004.1916.x. PMID: 15080818.
Kitson S L. 5-hydroxytryptamine (5-HT) receptor ligands. Curr Pharm Des. 007; 13(25):2621-37. doi: 10.2174/138161207781663000. PMID: 17897004.
Kushnir-Sukhov N M, Gilfillan A M, Coleman J W, Brown J M, Bruening S, Toth M, Metcalfe D D. 5-hydroxytryptamine induces mast cell adhesion and migration. J Immunol. 2006 Nov. 1; 177(9):6422-32. doi: 10.4049/jimmunol. 177.9.6422. PMID: 17056574.
Lechin F, van der Dijs B, Lechin A, Orozco B, Lechin M, Biez S, Rada I, Le6n G, Acosta E. Plasma neurotransmitters and cortisol in chronic illness: role of stress. J Med. 1994; 25(3-4):181-92. PMID: 7996062.
Lechin F, van der Dijs B, Orozco B, Lechin M, Lechin A E. Increased levels of free serotonin in plasma of symptomatic asthmatic patients. Ann Allergy Asthma Immunol. 1996 September; 77(3):245-53. doi: 10.1016/S1081-1206(10)63263-2. PMID: 8814052.
Lima C, Souza V M, Soares A L, Macedo M S, Tavares-de-Lima W, Vargaftig B B. Interference of methysergide, a specific 5-hydroxytryptamine receptor antagonist, with airway chronic allergic inflammation and remodeling in a murine model of asthma. Clin Exp Allergy. 2007 May; 37(5):723-34. doi: 10.1111/j.1365-2222.2007.02700.x. PMID: 17456220.
Meier M A, Ottiger M, Végeli A, Steuer C, Bernasconi L, Thomann R, Christ-Crain M, Henzen C, Hoess C, Zimmerli W, Huber A, Mueller B, Schuetz P. Activation of the Serotonin Pathway is Associated with Poor Outcome in COPD Exacerbation: Results of a Long-Term Cohort Study. Lung. 2017 June; 195(3):303-311. doi: 10.1007/s00408-017-0004-7. Epub 2017 Apr. 22. PMID: 28434116.
Ménard G, Turmel V, Bissonnette E Y. Serotonin modulates the cytokine network in the lung: involvement of prostaglandin E2. Clin Exp Immunol. 2007 November; 150(2):340-8. doi: 10.1111/j.1365-2249.2007.03492.x. Epub 2007 Sep. 5. PMID: 17822443; PMCID: PMC2219342.
Mims J W. Asthma: definitions and physiopathology. Int Forum Allergy Rhinol. 2015 September; 5 Suppl 1:S2-6. doi: 10.1002/alr.21609. PMID: 26335832.
Müller T, Dúrk T, Blumenthal B, Grimm M, Cicko S, Panther E, Sorichter S, Herouy Y, Di Virgilio F, Ferrari D, Norgauer J, Idzko M. 5-hydroxytryptamine modulates migration, cytokine and chemokine release and T-cell priming capacity of dendritic cells in vitro and in vivo. PLoS One. 2009 Jul. 31; 4(7):e6453. doi: 10.1371/journal.pone.0006453. PMID: 19649285; PMCID: PMC2714071.
Nichols D E, Nichols C D. Serotonin receptors. Chem Rev. 2008 May; 108(5):1614-41. doi: 10.1021/cr078224o. Epub 2008 May 14. PMID: 18476671.
Onclinx C. Relation entre la résistance pulmonaire totale et la Penh en fonction de la localisation anatomique de l'obstruction des voies aériennes (Master thesis). University of Liege, School of Veterinary Medicine: Liège, (2003), 24p.
Ortega H G, Liu M C, Pavord I D, Brusselle G G, FitzGerald J M, Chetta A, Humbert M, Katz L E, Keene O N, Yancey S W, Chanez P; MENSA Investigators. Mepolizumab treatment in patients with severe eosinophilic asthma. N Engl J Med. 2014 Sep. 25; 371(13):1198-207. doi: 10.1056/NEJMoa1403290. Epub 2014 Sep. 8. Erratum in: N Engl J Med. 2015 Apr. 30; 372(18):1777. PMID: 25199059.
Pakala R, Willerson J T, Benedict C R. Effect of serotonin, thromboxane A2, and specific receptor antagonists on vascular smooth muscle cell proliferation. Circulation. 1997 Oct. 7; 96(7):2280-6. doi: 10.1161/01.cir.96.7.2280. PMID: 9337201.
Pirina P, Zinellu E, Paliogiannis P, Fols A G, Marras V, Sotgia S, Carru C, Zinellu A. Circulating serotonin levels in COPD patients: a pilot study. BMC Pulm Med. 2018 Nov. 8; 18(1):167. doi: 10.1186/s12890-018-0730-5. PMID: 30409142; PMCID: PMC6225723.
Reddel H K, Bateman E D, Becker A, Boulet L P, Cruz A A, Drazen J M, Haahtela T, Hurd S S, Inoue H, de Jongste J C, Lemanske R F, Jr., Levy M L, O'Byrne P M, Paggiaro P, Pedersen S E, Pizzichini E, Soto-Quiroz M, Szefler S J, Wong G W, FitzGerald J M. A summary of the new GINA strategy: a roadmap to asthma control. Eur Respir J 2015; 46: 622-639.
Thomson N C, Bicknell S, Chaudhuri R. Bronchial thermoplasty for severe asthma. Curr Opin Allergy Clin Immunol. 2012 June; 12(3):241-8. doi: 10.1097/ACI.0b013e32835335ca. PMID: 22475996.

What is claimed is:

1. A method for inducing bronchodilation and/or preventing or reducing exacerbated bronchial reactivity in a mammal having an inflammatory respiratory disease, comprising administering to said mammal a therapeutically effective amount of a composition comprising at least one phytoecdysone and/or at least one semi-synthetic derivative of 20-hydroxyecdysone, said semi-synthetic derivative of 20-hydroxyecdysone being selected from the group consisting of:
a compound of general formula (I): 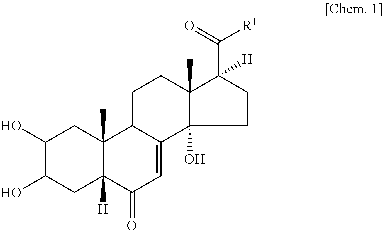
wherein:
R¹ is selected from the group consisting of: a (C₁-C₆)W(C₁-C₆) group; a (C₁-C₆)W(C₁-C₆)W(C₁-C₆) group; a (C₁-C₆)W(C₁-C₆)CO₂(C₁-C₆) group; a (C₁-C₆)A group, A representing a heterocycle optionally substituted by an OH, OMe, (C₁-C₆), N(C₁-C₆), or CO₂(C₁-C₆) type-group; a CH₂Br group;
W being a heteroatom selected from the group consisting of N, O and S; and,
a compound having formula (II):

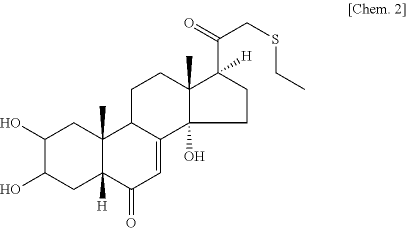 [SMILES: CCSCC(=O)[C@H]1CC[C@@]2(O)C3=CC(=O)[C@@H]4CC(O)C(O)C[C@]4(C)C3CC[C@]12C]

2. The method as claimed in claim 1, wherein the inflammatory respiratory disease is a chronic inflammatory respiratory disease.

3. The method as claimed in claim 1, wherein the inflammatory respiratory disease is asthma, chronic obstructive pulmonary disease or cystic fibrosis.

4. The method as claimed in claim 1, wherein the composition is used in the treatment of respiratory function impairment linked to the serotoninergic pathway in the mammal having an inflammatory respiratory disease.

5. The method as claimed in claim 4, wherein the composition is used in the treatment of respiratory function impairment linked to the serotoninergic pathway 5-HT_{1A} receptor in the mammal having an inflammatory respiratory disease.

6. The method as claimed in claim 1, wherein the composition comprises 20-hydroxyecdysone.

7. The method as claimed in claim 6, wherein 20-hydroxyecdysone is in the form of a plant extract or plant part, said plant being selected from the group consisting of plants containing at least 0.5% of 20-hydroxyecdysone by dry weight of said plant, said extract comprising at least 95% of 20-hydroxyecdysone.

8. The method as claimed in claim 7, wherein the extract comprises between 0 and 0.05%, by dry weight of the extract, of impurities capable of affecting the safety, availability or efficacy of a pharmaceutical application of said extract.

9. The method as claimed in claim 7, wherein the plant is selected from the group consisting of Stemmacantha carthamoides, Cyanotis arachnoidea, Cyanotis vaga and Pfaffia paniculata.

10. The method as claimed in claim 1, wherein in general formula (I):
R¹ is selected from the group consisting of: a (C₁-C₆)W(C₁-C₆) group; a (C₁-C₆)W(C₁-C₆)W(C₁-C₆) group; a (C₁-C₆)W(C₁-C₆)CO₂(C₁-C₆) group; a (C₁-C₆)A group, A representing a heterocycle optionally substituted by an OH, OMe, (C₁-C₆), N(C₁-C₆), CO₂(C₁-C₆) type-group;
W being a heteroatom selected from the group consisting of N, O and S.

11. The method as claimed in claim 1, wherein said compound of general formula (I) is selected from the group consisting of:
No. 1: (2S,3R,5R,10R,13R,14S,17S)-2,3,14-trihydroxy-10.13-dimethyl-17-(2-morpholinoacetyl)-2,3,4,5,9,11,12,15,16,17-decahydro-1H-cyclopenta[a]phenanthren-6-one;
No. 2: (2S,3R,5R,10R,13R,14S,17S)-2,3,14-trihydroxy-17-[2-(3-hydroxypyrrolidin-1-yl)acetyl]-10,13-dimethyl-2,3,4,5,9,11,12,15,16,17-decahydro-1H-cyclopenta[a]phenanthren-6-one;
No. 3: (2S,3R,5R,10R,13R,14S,17S)-2,3,14-trihydroxy-17-[2-(4-hydroxy-1-piperidyl)acetyl]-10,13-dimethyl-2,3,4,5,9,11,12,15,16,17-decahydro-1H-cyclopenta[a]phenanthren-6-one;
No. 4: (2S,3R,5R,10R,13R,14S,17S)-2,3,14-trihydroxy-17-[2-[4-(2-hydroxyethyl)-1-piperidyl]acetyl]-10,13-dimethyl-2,3,4,5,9,11,12,15,16,17-decahydro-1H-cyclopenta[a]phenanthren-6-one;
No. 5: (2S,3R,5R,10R,13R,14S,17S)-17-[2-(3-dimethylaminopropyl(methyl)amino)acetyl]-2,3,14-trihydroxy-10,13-dimethyl-2,3,4,5,9,11,12,15,16,17-decahydro-1H-cyclopenta[a]phenanthren-6-one;
No. 6: 2-[2-oxo-2-[(2S,3R,5R,10R,13R,14S,17S)-2,3,14-trihydroxy-10,13-dimethyl-6-oxo-2,3,4,5,9,11,12,15,16,17-decahydro-1H-cyclopenta[a]phenanthren-17-yl]ethyl]ethyl sulfanylacetate;
No. 7: (2S,3R,5R,10R,13R,14S,17S)-17-(2-ethylsulfanylacetyl)-2,3,14-trihydroxy-10.13-dimethyl-2,3,4,5,9,11,12,15,16,17-decahydro-1H-cyclopenta[a]phenanthren-6-one;
No. 8: (2S,3R,5R,10R,13R,14S,17S)-2,3,14-trihydroxy-17-[2-(2-hydroxyethylsulfanyl)acetyl]-10,13-dimethyl-2,3,4,5,9,11,12,15,16,17-decahydro-1H cyclopenta[a]phenanthren-6-one.

12. The method as claimed in claim 7, wherein said extract comprises at least 97% of 20-hydroxyecdysone.
